(19) United States
(12) Patent Application Publication
(10) Pub. No.: US 2021/0250473 A1
(43) Pub. Date: Aug. 12, 2021

(54) IMAGE PICKUP APPARATUS, ENDOSCOPE AND METHOD FOR MANUFACTURING IMAGE PICKUP APPARATUS

(71) Applicant: OLYMPUS CORPORATION, Tokyo (JP)

(72) Inventor: Takuro SUYAMA, Ina-shi (JP)

(73) Assignee: OLYMPUS CORPORATION, Tokyo (JP)

(21) Appl. No.: 17/241,685

(22) Filed: Apr. 27, 2021

Related U.S. Application Data
(63) Continuation of application No. PCT/JP2018/040055, filed on Oct. 29, 2018.

(51) Int. Cl. H04N 5/225; H01L 27/146

(52) U.S. Cl. CPC H04N 5/2253; H04N 5/2254; H04N 2005/2255; H01L 27/14685; H01L 27/14625

(57) ABSTRACT

An image pickup apparatus includes: a wiring board including a mounting surface; an image pickup device mounted on the mounting surface, the image pickup device including, on a light-receiving surface thereof, a light-receiving section; an optical component including a first main surface and a second main surface, the second main surface being bonded to the light-receiving surface; an electronic component mounted on the mounting surface; and sealing resin disposed on the mounting surface, covering the image pickup device, located on the same surface as the first main surface and including a resin upper surface parallel to the mounting surface.

### CROSS REFERENCE TO RELATED APPLICATION

This application is a continuation application of PCT/JP2018/040055 filed on Oct. 29, 2018, the entire contents of which are incorporated herein by this reference.

### BACKGROUND OF THE INVENTION

### 1. Field of the Invention

The present invention relates to an image pickup apparatus, an image pickup section of which is sealed with sealing resin, an endoscope including an image pickup apparatus, an image pickup section of which is sealed with sealing resin and a method for manufacturing an image pickup apparatus, an image pickup section of which is sealed with sealing resin.

### 2. Description of the Related Art

Electronic endoscopes equipped with an image pickup apparatus at a distal end portion of an insertion portion have come into widespread use. Medical endoscopes perform observation and treatment of a site to be examined by inserting a flexible elongated insertion portion with an image pickup apparatus incorporated at a distal end portion into a body cavity of a subject such as a patient.

Japanese Patent Application Laid-Open Publication No. 2015-198726 discloses an endoscope including a horizontally set image pickup apparatus. In the horizontally set image pickup apparatus, a prism is bonded to a light-receiving surface of an image pickup device.

Japanese Patent Application Laid-Open Publication No. 2009-170469 discloses an image pickup apparatus with a side surface of a cover glass of an image pickup device covered with black resin for light shielding.

### SUMMARY OF THE INVENTION

An image pickup apparatus according to an embodiment includes a wiring board including a mounting surface, an image pickup component mounted on the mounting surface, a light-receiving section being disposed on a light-receiving surface of the image pickup component, an optical component including a first main surface and a second main surface on an opposite side of the first main surface, the second main surface being bonded to the light-receiving surface, an electronic component mounted on the mounting surface, the electronic component being a semiconductor device, a passive device, or an illumination component and sealing resin disposed on the mounting surface, covering the image pickup component, located on a same surface as the first main surface and including a resin upper surface parallel to the mounting surface.

An endoscope according to the embodiment includes an image pickup apparatus, the image pickup apparatus including a wiring board including a mounting surface, an image pickup component mounted on the mounting surface, a light-receiving section being disposed on a light-receiving surface of the image pickup component, an optical component including a first main surface and a second main surface on an opposite side of the first main surface, the second main surface being bonded to the light-receiving surface, an electronic component mounted on the mounting surface, the electronic component being a semiconductor device, a passive device, or an illumination component and sealing resin disposed on the mounting surface, covering the image pickup component, located on a same surface as the first main surface and including a resin upper surface parallel to the mounting surface.

A method for manufacturing an image pickup apparatus according to the embodiment includes mounting an image pickup component with an optical component bonded to a light-receiving surface, and an electronic component, which is a semiconductor device, a passive device, or an illumination component, on a mounting surface of a wiring board, disposing sealing resin on the mounting surface, the sealing resin covering the image pickup component and the optical component, and polishing the sealing resin to thereby expose the optical component to a polished surface and polishing the optical component exposed to the polished surface and the sealing resin simultaneously.

### BRIEF DESCRIPTION OF THE DRAWINGS

FIG. 1 is an external view of an endoscope system including an endoscope according to an embodiment;

FIG. 2 is a cross-sectional view of an image pickup apparatus according to a first embodiment;

FIG. 3 is a flowchart for describing a method for manufacturing an image pickup apparatus of the first embodiment;

FIG. 4 is a cross-sectional view for describing the method for manufacturing an image pickup apparatus of the first embodiment;

FIG. 5 is a cross-sectional view for describing the method for manufacturing an image pickup apparatus of the first embodiment;

FIG. 6 is a cross-sectional view for describing the method for manufacturing an image pickup apparatus of the first embodiment;

FIG. 7 is a cross-sectional view for describing the method for manufacturing an image pickup apparatus of the first embodiment;

FIG. 8 is a cross-sectional view of an image pickup apparatus according to a modification 1 of the first embodiment;

FIG. 9 is a cross-sectional view for describing a method for manufacturing an image pickup apparatus according to a modification 2 of the first embodiment;

FIG. 10 is a cross-sectional view for describing a method for manufacturing an image pickup apparatus according to a modification 3 of the first embodiment;

FIG. 11 is a cross-sectional view of an image pickup apparatus according to a second embodiment;

FIG. 12 is a cross-sectional view for describing a method for manufacturing an image pickup apparatus according to the second embodiment; and

FIG. 13 is a cross-sectional view of an image pickup apparatus according to a modification of the second embodiment.

### DETAILED DESCRIPTION OF THE PREFERRED EMBODIMENTS

### <Configuration of Endoscope>

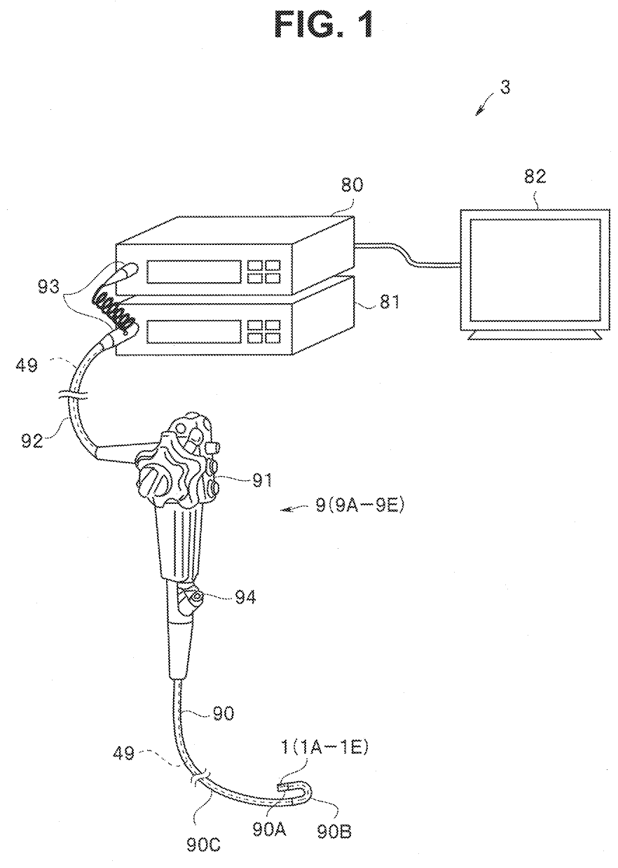

As illustrated in FIG. 1, an endoscope system 3 is provided with an endoscope 9 according to an embodiment, a processor 80, a light source apparatus 81 and a monitor 82. For example, with a flexible insertion portion 90 inserted into a body cavity of a subject, the endoscope 9 shoots an in-vivo image of the subject and outputs an image pickup signal.

An operation portion (intermediate portion) 91 provided with various buttons for operating the endoscope 9 is disposed at a proximal end portion of the insertion portion 90 of the endoscope 9. The operation portion 91 is provided with a treatment instrument insertion port of a channel 94 through which a bio forceps, an electric scalpel, an inspection probe or the like is inserted into the body cavity of the subject.

The insertion portion 90 is constructed of a rigid distal end portion 90A at which an image pickup apparatus 1 is disposed, a freely bendable bending portion 90B connected to a proximal end portion of the distal end portion 90A and a flexible portion 90C connected to a proximal end portion of the bending portion 90B. The bending portion 90B is bent by operation of the operation portion 91. The image pickup apparatus 1 disposed at the distal end portion 90A transmits image pickup signals via a plurality of signal cables 49.

A universal cord 92 extending from the operation portion 91 is connected to the processor 80 and the light source apparatus 81 via a connector 93.

The processor 80 controls the entire endoscope system 3, and also performs signal processing on an image pickup signal and outputs the image pickup signal as an image signal. The monitor 82 displays the image signal outputted from the processor 80.

The light source apparatus 81 includes, for example, a white LED. Illumination light emitted from the light source apparatus 81 is guided to an illumination optical system (not shown) of the distal end portion 90A via a light guide (not shown) that is inserted through the universal cord 92 and the insertion portion 90 and illuminates an object.

As will be described later, the image pickup apparatus 1 shows high reliability, and therefore the endoscope 9 shows high reliability.

Note that the endoscope 9 may be a rigid endoscope or a capsule type endoscope. The endoscope 9 may be used for medical or industrial purposes.

### First Embodiment

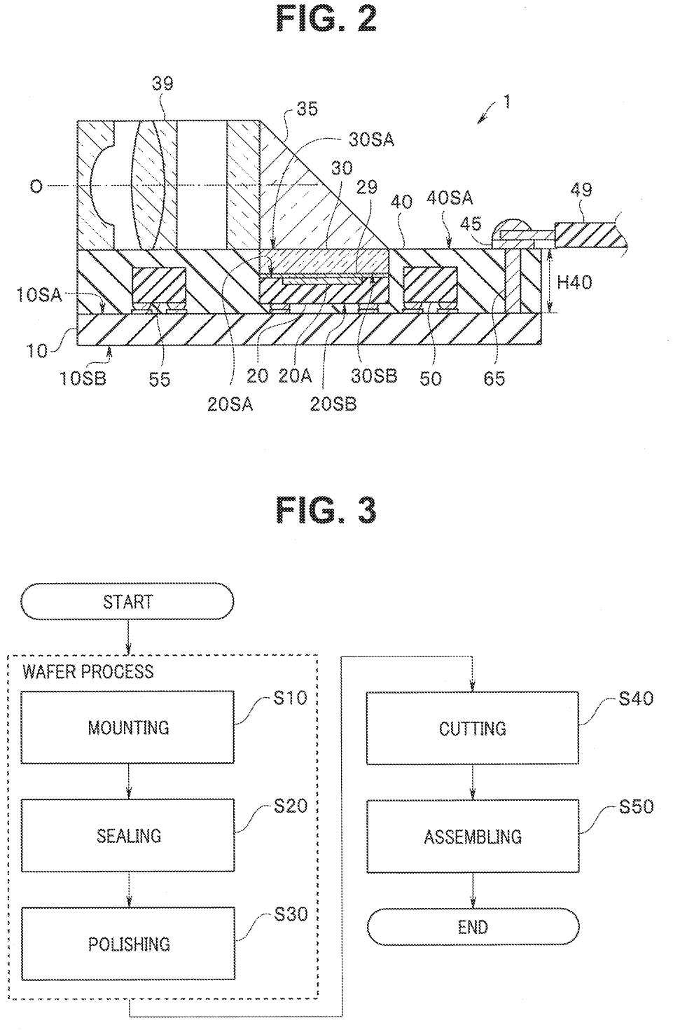

As illustrated in FIG. 2, the image pickup apparatus 1 of the present embodiment is of a so-called “horizontally set” type where an optical axis O of a lens unit 39, which is an objective optical system, is parallel to a light-receiving surface 20SA of an image pickup device 20, which is an image pickup section.

In the following description, note that the drawings are schematic, and a thickness-width relationship in the respective components, a thickness ratio or the like among the respective components are different from real components, and components having different dimensional relationships or ratios among the drawings may be included.

The image pickup apparatus 1 is provided with a wiring board 10, an image pickup device 20, a cover glass 30, which is an optical section, electronic components 50 and 55, sealing resin 40 and a prism 35.

The wiring board 10 includes a mounting surface 10SA and an opposite surface 10SB on an opposite side of the mounting surface 10SA. The image pickup device 20 includes a light-receiving surface 20SA and a reverse surface 20SB on an opposite side of the light-receiving surface 20SA, and a light-receiving section 20A is disposed on the light-receiving surface 20SA. In the image pickup device 20, the reverse surface 20SB is disposed so as to face the mounting surface 10SA and electrically connected to an electrode (not shown) of the mounting surface 10SA. In other words, the image pickup device 20 is electrically mounted to the mounting surface 10SA.

The cover glass 30 includes a first main surface 30SA and a second main surface 30SB on an opposite side of the first main surface 30SA, and is a planar component, which is a transparent material. The second main surface 30SB is bonded to the light-receiving surface 20SA using a transparent bonding layer 29 so as to cover the light-receiving section 20A. Note that a transparent resin plate, for example, may also be provided instead of the cover glass 30.

The electronic components 50 and 55, such as chip capacitors, are mounted on the mounting surface 10SA.

The sealing resin 40 is disposed on the mounting surface 10SA and covers the image pickup device 20 and the electronic components 50 and 55. The sealing resin 40 includes a resin upper surface 40SA. The resin upper surface 40SA is the same surface as the first main surface 30SA and is parallel to the mounting surface 10SA. In other words, a side surface of the cover glass 30 is covered with the sealing resin 40, whereas the first main surface 30SA is an exposed surface, which is not covered with the sealing resin 40. Since the resin upper surface 40SA and the first main surface 30SA are polished surfaces, which have been polished simultaneously, there is no height difference between both the surfaces and the both surfaces constitute one surface parallel to the mounting surface 10SA.

The sealing resin 40 that improves humidity resistance of the image pickup device 20 and the electronic components 50 and 55 is thermosetting resin with low water vapor permeability such as epoxy resin, acrylic resin or styrene resin. The sealing resin 40 is preferably a rigid material to secure mechanical strength of the image pickup apparatus 1.

Furthermore, the sealing resin 40 is preferably light shielding resin with low light transmission in which light-shielding particles are dispersed. The image pickup device 20 sealed with the light shielding resin is less susceptible to external light.

For example, the sealing resin 40 preferably exhibits a water vapor permeability of 50 g/(m²×day) or less in a water vapor permeability test defined in JIS Z 0208. Furthermore, the sealing resin 40 preferably exhibits a Vickers hardness Hv of 40 or higher, and particularly preferably exhibits 100 or higher. In accordance with JIS Z2244, hardness Hv is measured using a Vickers hardness meter from an indentation generated when the sealing resin 40 is inserted into a sample having a weight of 1 kg under a condition of 25° C. and a pressing time of 15 seconds.

Note that as will be described later, the resin upper surface 40SA of the sealing resin 40 and the first main surface 30SA of the cover glass 30 are polished surfaces resulting from a CMP (chemical mechanical polishing) process. In other words, the surfaces constituted by the resin upper surface 40SA and the first main surface 30SA are surfaces simultaneously polished by a polishing process.

The first main surface 30SA of the cover glass 30 preferably exhibits less surface roughness than the surface roughness of the resin upper surface 40SA particularly from the standpoint of optical characteristic and adhesiveness.

The horizontally set image pickup apparatus 1 is further provided with the prism 35, the lens unit 39 and the plurality of signal cables 49. The prism 35 is bonded to the first main surface 30SA of the cover glass 30 via the transparent bonding layer 29.

Note that there can also be a case where the bonding surface (light emitting surface) of the prism 35 bonded to the first main surface 30SA of the cover glass 30 has a larger size and a larger area than the size and the area of the first main surface 30SA of the cover glass 30. In other words, the light emitting surface of the prism 35 may project from the first main surface 30SA. However, since the first main surface 30SA is the same surface as the surrounding resin upper surface 40SA, the prism 35 is accurately disposed.

Though not shown in detail, the lens unit 39 includes a plurality of lenses, a spacer, an aperture and a filter, and condenses an object image. The object image condensed by the lens unit 39 is incident on the prism 35. An optical path of the object image is bent 90 degrees by the prism 35, and the object image passes through the cover glass 30 and is incident on the light-receiving section 20A of the image pickup device 20.

The image pickup device 20 in the image pickup apparatus 1 is an imager made of a semiconductor such as silicon. The light-receiving section 20A is a CMOS (complementary metal oxide semiconductor) type semiconductor light-receiving circuit or a CCD (charge coupled device). A plurality of electrodes electrically connected to the light-receiving section 20A are disposed on the reverse surface 20SB of the image pickup device 20. The electrode of the image pickup device 20 is joined to the electrode on the mounting surface 10SA of the wiring board 10, and thereby electrically connected. Note that the electrode disposed on the light-receiving surface 20SA may be connected to the electrode of the mounting surface 10SA of the wiring board 10 via a bonding wire.

A plurality of junction electrodes 45 disposed on the resin upper surface 40SA are electrically connected to the light-receiving section 20A and the electronic components 50 and 55 via respective through wirings 65, which are respectively connected to the wiring board 10. The image pickup signal outputted from the image pickup device 20 is transmitted to the plurality of signal cables 49 through the plurality of electrodes on the mounting surface 10SA of the wiring board 10, the plurality of through wirings 65 and the plurality of junction electrodes 45.

The electronic components 50 and 55 are semiconductor devices or passive devices for signal processing, and part of an image pickup signal may pass through the electronic components 50 and 55. The number of electronic components mounted on the wiring board 10 may be one or three or more. The image pickup apparatus 1 including the electronic components has good image pickup characteristics.

Since the image pickup device 20 and the electronic components 50 and 55 are sealed with the sealing resin 40 with low water vapor permeability, the image pickup apparatus 1 is highly reliable. Moreover, since the thin wiring board 10 is reinforced by the sealing resin 40, the image pickup apparatus 1 is small and even if stress is applied to the image pickup apparatus 1, it is less likely that the wiring board 10 or the image pickup device 20 may be damaged or junction reliability of the image pickup device 20 may deteriorate. For this reason, the image pickup apparatus 1 and the endoscope 9 including the image pickup apparatus 1 are small and highly reliable.

### <Method for Manufacturing Image Pickup Apparatus>

Next, a method for manufacturing the image pickup apparatus 1 will be described according to a flowchart in FIG. 3. In the image pickup apparatus 1, a main part 2 (see FIG. 7 or the like) will be manufactured through a wafer process, the main part 2 having a configuration in which the image pickup device 20 to which the cover glass 30 is bonded and the electronic components 50 and 55 are mounted on the mounting surface 10SA of the wiring board 10, and the cover glass 30, the image pickup device 20 and the electronic components 50 and 55 are sealed with the sealing resin 40.

### <Step S10> Mounting Step

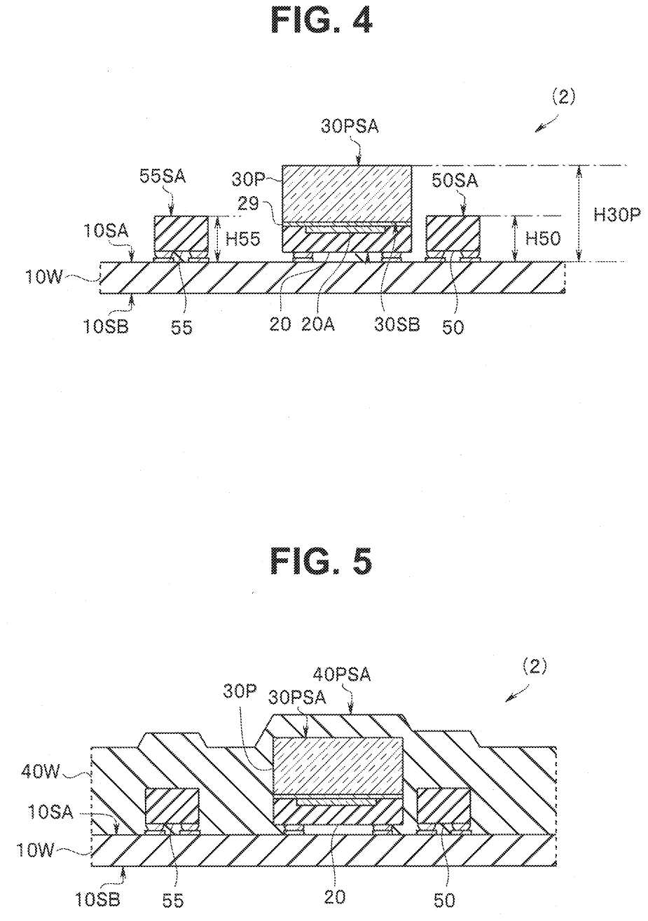

As illustrated in FIG. 4, a configuration (2), which becomes the main part 2 of the image pickup apparatus 1, is manufactured, in which the electronic components 50 and 55 and the image pickup device 20, which is a chip size package, to which a glass chip 30P is bonded are mounted on the mounting surface 10SA of a wiring wafer 10W, which becomes the wiring board 10. For example, when the glass chip 30P is processed in a polishing step, the glass chip 30P becomes the cover glass 30 of the main part 2.

The wiring wafer 10W is based on a resin substrate, a ceramic substrate, a glass epoxy substrate, a glass substrate or a silicon substrate. The wiring wafer 10W is preferably based on a thin flexible resin substrate to reduce the size of the image pickup apparatus 1. The thin wiring wafer 10W is used by being pasted to a rigid support wafer. By disposing a wiring layer including a wire and an insulating layer on the rigid support wafer and separating the support wafer in a later step, it is also possible to use the wiring layer as the wiring wafer 10W. Although the wiring layer separated from the support wafer is quite thin, the strength of the wiring layer is guaranteed by a sealing resin 40W.

The image pickup device 20 covered with the glass chip 30P is manufactured by a wafer process. In other words, an image pickup wafer for which a plurality of light-receiving sections 20A or the like are formed is manufactured using known semiconductor techniques. A glass wafer is bonded to the image pickup wafer using the transparent bonding layer 29. By dividing the image pickup wafer to which the glass wafer is bonded into individual pieces, the image pickup device 20, the light-receiving surface 20SA of which is covered with the glass chip 30P, is manufactured.

Examples of the electronic components 50 and 55 include chip capacitors, chip inductors, chip resistors or integrated circuits. These electronic components perform primary signal processing and amplification or the like on an image pickup signal outputted from the image pickup device 20.

Note that a height H30P from the mounting surface 10SA to a glass upper surface 30PSA of the glass chip 30P is larger than heights H50 and H55 from the mounting surface 10SA to upper surfaces 50SA and 55SA of the electronic components 50 and 55.

### <Step S20> Sealing Step

As illustrated in FIG. 5, the uncured sealing resin 40W is disposed on the mounting surface 10SA of the wiring wafer 10W and a thermosetting process is carried out. Entire surfaces (upper surface and side surfaces) of the image pickup device 20, the glass chip 30P and the electronic components 50 and 55 are covered with the sealing resin 40W.

### <Step S30> Polishing Step

A polishing step S30 includes a first polishing step and a second polishing step.

In the first polishing step, a resin upper surface 40PSA of the sealing resin 40W on the wiring wafer 10W (see FIG. 5) is polished. By polishing the sealing resin 40W, the glass upper surface 30PSA of the glass chip 30P is exposed to the polished surface.

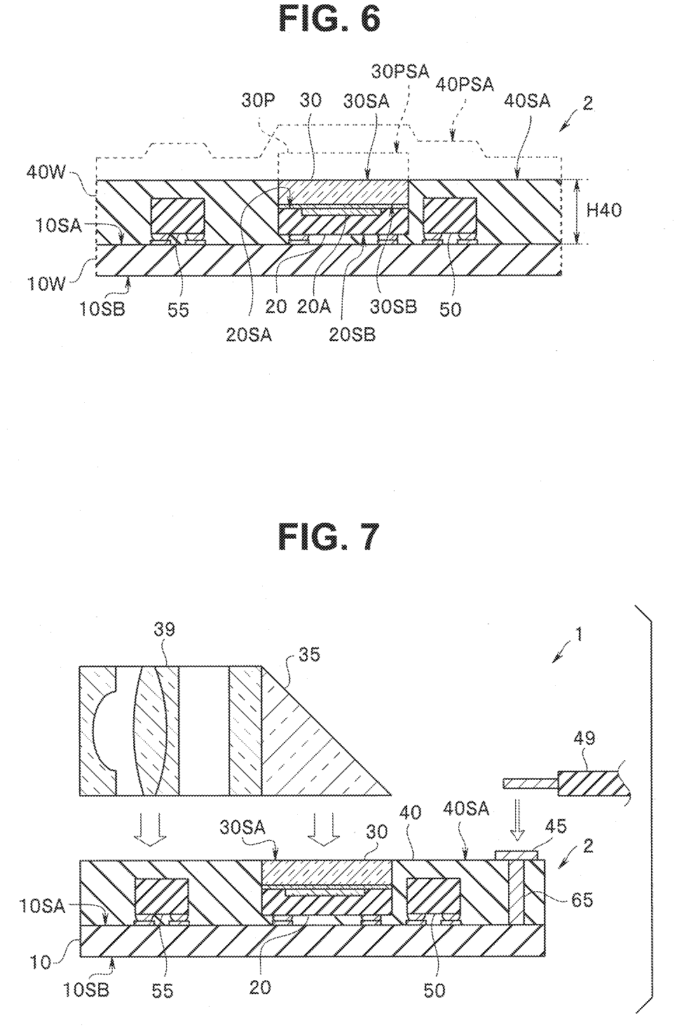

As illustrated in FIG. 6, in the second polishing step, the sealing resin 40W and the glass chip 30P are simultaneously polished.

In the second polishing step, when the sealing resin 40W is polished to a prescribed thickness of H40, the glass chip 30P becomes the cover glass 30, the first main surface 30SA of which is a mirror surface. The resin upper surface 40SA is located on the same surface as the first main surface 30SA and the resin upper surface 40SA and the first main surface 30SA are parallel to the mounting surface 10SA of the wiring wafer 10W.

Since the heights H50 and H55 of the electronic components 50 and 55 are smaller than the thickness H40, the entire electronic components 50 and 55 remain covered with the sealing resin 40W even after the second polishing step.

In the second polishing step, it is preferable to use a CMP process on condition that the polished surface is mirrored and the polishing speed of the sealing resin 40W and the polishing speed of the glass chip 30P are the same. In the first polishing step, the same CMP process as the CMP process in the second polishing step may be used or a mechanical polishing process may be used.

Next, a through wiring 65 that penetrates the sealing resin 40W is disposed and a junction electrode 45 is disposed on the through wiring 65.

The through wiring 65 is disposed by forming a through hole in the sealing resin 40 by etching using an etching mask patterned using a photolithography method or by laser processing or the like, and disposing a conductor in the through hole using a plating method. Note that the through wiring 65 may be disposed by disposing a column in the wiring wafer 10W using a plating method before the sealing step, disposing a column made of a conductive material and exposing the column upper surface of the column in the polishing step. The through wiring 65 is made of a conductive material such as titanium, nickel, copper, silver, aluminum or gold.

The junction electrode 45 connected to the through wiring 65 is joined to the signal cables 49. The junction electrode 45 is disposed, for example, by a pattern plating method, patterning of a sputtering film or a direct drawing method using a conductive paste or the like. The junction electrode 45 is made of a conductive material such as titanium, nickel, copper, silver, aluminum or gold.

### <Step S40> Cutting Step

Though not shown, the main part 2 is manufactured by cutting the wiring wafer 10W on which a plurality of image pickup devices 20, a plurality of cover glasses 30, a plurality of electronic components 50 and 55 and a sealing resin 41W are disposed. The main part 2 is constructed of the image pickup device 20, the cover glass 30 and the electronic components 50 and 55, which are mounted on the mounting surface 10SA of the wiring board 10 and sealed with the sealing resin 40.

### <Step S50> Assembling Step

As illustrated in FIG. 7, the prism 35 and the lens unit 39 are disposed on the main part 2. Since the electronic component 55 is disposed below the lens unit 39, the image pickup apparatus 1 is small.

The signal cables 49 are joined to the junction electrode 45. For example, a flexible wiring board may be disposed between the junction electrode 45 and the signal cable 49. In other words, a first electrode of the flexible wiring board may be joined to the junction electrode 45 and an electric cable may be bonded to a second electrode connected to the first electrode.

Since the wiring board 10 is reinforced by the sealing resin 40, the mechanical strength of the image pickup apparatus 1 is guaranteed. For this reason, even when stress is applied to the wiring board 10, there is no risk of the wiring board 10 or the image pickup device 20 being damaged, or due to deformation of the wiring board 10, there is no risk of degradation in junction reliability in the electrical connection portions in the main part 2, for example, between the electrode of the image pickup device 20 and the electrode on the mounting surface 10SA of the wiring board 10, between the electronic device and the wiring board, between the through wiring and the wiring board, or between the through wiring and the junction electrodes for cable connection. Since the main part 2 is manufactured by a wafer process, the image pickup apparatus 1 can be manufactured easily.

### Modifications of First Embodiment

Image pickup apparatuses 1A, 1B and 1C according to modifications of the first embodiment have configurations similar to the configuration of the image pickup apparatus 1 according to the first embodiment and have the same effects as the effects of the image pickup apparatus 1, and so components having the same functions are assigned the same reference numerals and description will be omitted.

### Modification 1 of First Embodiment

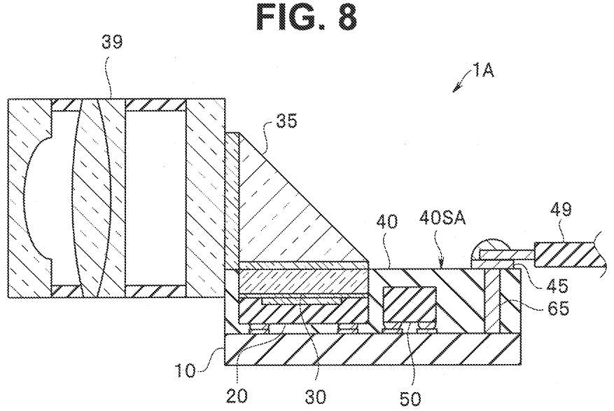

In the image pickup apparatus 1A according to the present modification illustrated in FIG. 8, a lens unit 39A is not disposed on the main part 2. In order to obtain good image pickup performance, after bonding the prism 35 to the main part 2, the large lens unit 39A is bonded to the prism 35 using a different transparent adhesive.

### Modification 2 of First Embodiment

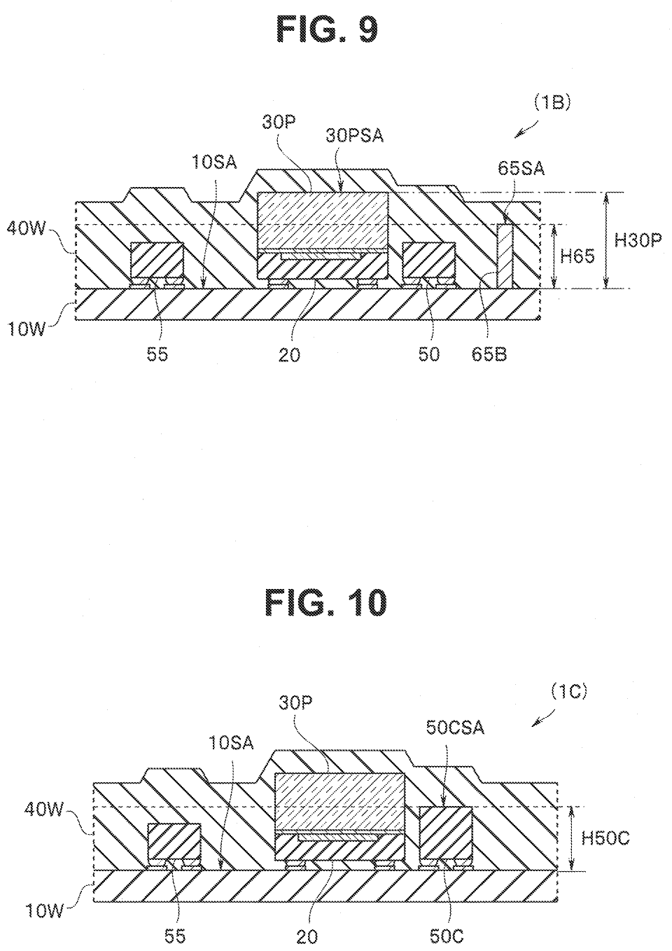

In the image pickup apparatus 1B according to the present modification, a plurality of through wirings that penetrate the sealing resin 40 are constructed of a plurality of copper columns 65B. As illustrated in FIG. 9, in a mounting step S10, the columns 65B are mounted on the mounting surface 10SA of the wiring wafer 10W. A height H65 of the columns 65B is slightly larger than the thickness of the sealing resin 40W after polishing and is set to less than the height H30P up to the glass upper surface 30PSA of the glass chip 30P. More specifically, the height H65 of the columns 65B is set as appropriate in consideration of a polishing speed.

The polishing speed of CMP for each material varies depending on polishing conditions. Under the polishing condition according to the modification 2, the polishing speed of CMP for the columns 65B is quite slow compared to the sealing resin 40W and the glass chip 30P. For example, the polishing speed for the copper columns 65B is preferably ⅕ or less of the polishing speed for the sealing resin 40W and the glass chip 30P, and particularly more preferably 1/10 or less.

In the second polishing step, when the sealing resin 40W and the glass chip 30P are polished and column upper surfaces 65SA of the columns 65B are exposed, the polishing speed for the sealing resin 40W and the glass chip 30P become very slow and the polishing step is substantially finished.

In the image pickup apparatus 1B, the height H65 of the copper columns 65B is substantially the same as an addition value of the thickness of the cover glass 30 and the thickness of the image pickup device 20 and the thickness of the sealing resin 40, and so it is easy to process the sealing resin 40W and the glass chip 30P to a prescribed thickness. Moreover, the step of disposing the through wiring after the polishing step is unnecessary. For this reason, the image pickup apparatus 1B is manufactured more easily than the image pickup apparatus 1.

### Modification 3 of First Embodiment

In the image pickup apparatus 1C according to the present modification, as illustrated in FIG. 10, in the mounting step S10, an electronic component 50C is mounted on the mounting surface 10SA of the wiring wafer 10W. A height H50C from a mounting surface 10SA to a component upper surface 50CSA of the electronic component 50C is set to a thickness of the sealing resin 40W after polishing.

The component upper surface 50CSA of the electronic component 50C is made of a material such as silicon, the polishing speed of CMP under the condition of the present modification of which is very slow compared to the sealing resin 40W and the glass chip 30P. For this reason, in the second polishing step, when the sealing resin 40W and the glass chip 30P are polished and the component upper surface 50CSA of the electronic component 50C is exposed, the polishing speed becomes very slow and the polishing step is substantially finished.

In the image pickup apparatus 1C, it is easy to process the sealing resin 40W and the glass chip 30P to a prescribed thickness. For this reason, the image pickup apparatus 1C is manufactured more easily than the image pickup apparatus 1.

As described so far, in the image pickup apparatuses 1B and 1C according to the modifications, the second polishing step is finished when the column upper surface 65BSA of the columns 65B made of the conductive material that penetrates the sealing resin 40 or the component upper surface 50CSB of the electronic component 50C is exposed.

Note that in the image pickup apparatus 1C of the present modification, the height of the electronic component 55 may be the same as the height of the electronic component 50C or the image pickup apparatus 1C may not include the electronic component 55.

### Second Embodiment

An image pickup apparatus 1D according to a second embodiment is similar to the image pickup apparatus 1 according to the first embodiment and has the same effects as the effects of the image pickup apparatus 1 according to the first embodiment, and so components having the same functions are assigned the same reference numerals and description will be omitted.

As illustrated in FIG. 11, in the image pickup apparatus 1D, the optical section is a lens unit 39D including the cover glass 30. An optical axis O of the lens unit 39D, which is an objective optical system, is disposed perpendicular to the light-receiving surface 20SA of the image pickup device 20. The so-called “vertically set” image pickup apparatus 1D includes no prism.

The lens unit 39D is bonded to the cover glass 30 via a transparent bonding layer (not shown). If seen from another perspective, the optical section of the image pickup apparatus 1D is the lens unit 39D including the cover glass 30 and a plurality of lenses or the like, and the first main surface is an incident surface 39SA of the lens unit 39D. A forefront optical element (lens) that constitutes the incident surface 39SA is a lens made of glass or a resin material, which is a transparent material.

The image pickup apparatus 1D includes illumination sections 65 (65A, 65B), which are electronic components mounted on the mounting surface 10SA of a wiring board 10D. The illumination sections 65 (65A, 65B) are further provided with light-emitting elements 60 (60A, 60B) and light guide sections 70 (70A, 70B). Note that when a plurality of identical components are each referred to, the last character is omitted hereinafter. For example, a plurality of light-emitting elements 60A and 60B will each be referred to as a “light-emitting element 60.”

The light-emitting element 60 including a light-emitting surface 60SA and a rear surface 60SB on an opposite side of the light-emitting surface 60SA is, for example, an LED and generates illumination light from a light-emitting section 61. The light guide section 70 includes a third main surface 70SA and a fourth main surface 70SB on an opposite side of the third main surface 70SA. As for the light guide section 70, a material different from the material of the incident surface 39SA of the lens unit may be used as long as the polishing speed is substantially the same, whereas it is preferable to use the same transparent material such as glass. The fourth main surface 70SB of the light guide section 70 is bonded to the light-emitting surface 60SA of the light-emitting element 60 via a transparent bonding layer (not shown). The light guide section 70 guides illumination light and emits the illumination light from the third main surface 70SA. The light guide section 70 may include a lens function.

The light-emitting element 60 is different in thickness from the image pickup device 20, and so the light-emitting surface 60SA of the light-emitting element 60 is on a different surface from the light-receiving surface 20SA of the image pickup device 20. The third main surface 70SA of the light guide section 70 is on the same surface as the first main surface (incident surface) 39SA of the lens unit 39D and the resin upper surface 40SA of the sealing resin 40D, and is an exposed surface parallel to the mounting surface 10SA. A height H40D from the mounting surface 10SA to the resin upper surface 40SA is the same as the height to the first main surface (incident surface) 39SA of the lens unit 39D and the height to the third main surface 70SA of the light guide section 70.

There is no height difference among the third main surface 70SA, the resin upper surface 40SA and the first main surface (incident surface 39SA), and these surfaces constitute one surface. Note that in the image pickup apparatus 1D, the sealing resin 40 is highly preferably light shielding resin so as to prevent illumination light from being received by the light-receiving section 20A.

A plurality of junction electrodes 45D to which the light-receiving section 20A or the light-emitting section 61 is connected are disposed on the opposite surface 10SB of the wiring board 10D. The junction electrodes 45D are connected to a power supply section and a post-stage circuit section, for example, via a flexible wiring board.

The image pickup apparatus 1D in which the illumination section 65 is disposed around the image pickup device 20 can significantly contribute to a size reduction and reliability of a capsule type endoscope in particular. The image pickup apparatus 1D, the optical section of which is the lens unit 39D where a plurality of optical elements including the cover glass 30 are stacked, can be miniaturized and manufactured easily.

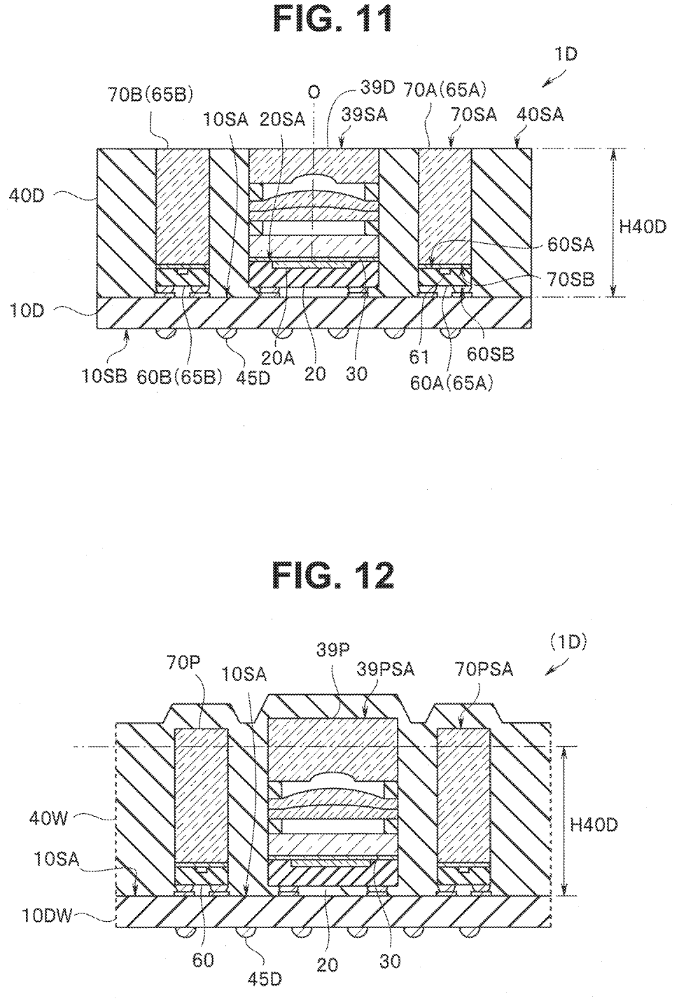

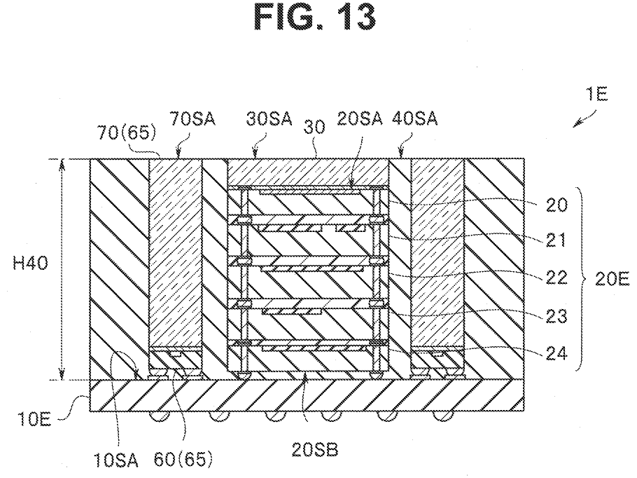

As illustrated in FIG. 12, according to the method for manufacturing the image pickup apparatus 1D, in the mounting step S10, the light-emitting element 60 to which the light guide section 70P is bonded and the image pickup device 20 to which the lens unit 39P including the cover glass 30 is bonded are disposed on the mounting surface 10SA of a wiring wafer 10DW. Note that a glass upper surface 70PSA of the light guide section 70P and a glass upper surface 39PSA of the lens unit 39P are different in height from the mounting surface 10SA. For example, the glass upper surface 39PSA of the lens unit 39P is higher than the glass upper surface 70PSA of the light guide section 70P.

In the sealing step S20, the light guide section 70P is also covered with the sealing resin 40W. In the polishing step S30, the light guide section 70P is polished and thereby becomes the light guide section 70 including the light emitting surface 70SA, which is an exposed surface.

In the polishing step of the image pickup apparatus 1D, in the first polishing step, only the sealing resin 40W is polished and the glass upper surface 39PSA of the lens unit 39P is exposed. In the second polishing step, the sealing resin 40W and the lens unit 39P are polished and the glass upper surface 70PSA of the light guide section 70P is exposed. Further polishing brings forth a state illustrated in FIG. 11. It goes without saying that when the glass upper surface 39PSA of the lens unit 39P is lower than the glass upper surface 70PSA of the light guide section 70P, the glass of the lens unit 39P is polished after the glass of the light guide section 70P is exposed.

Note that the electronic components mounted around the image pickup device 20 may be semiconductor devices that process an image signal from the image pickup device 20 or may be passive components that constitute an image pickup circuit.

### Modification of Second Embodiment

As illustrated in FIG. 13, in an image pickup apparatus 1E according to the present modification, an image pickup section 20E is a stacked semiconductor in which a plurality of semiconductor devices 20 to 24 including the image pickup device 20 are stacked.

The semiconductor devices 21 to 24 each include a semiconductor circuit such as an AD conversion processing circuit to perform primary processing on an image pickup signal outputted from a light-receiving section 20A, for example. The semiconductor devices 21 to 24 may include a transfer buffer, a resistor or a capacitor. The number of semiconductor devices and a type of the semiconductor circuit or the like incorporated are set according to the specifications of the image pickup apparatus. Semiconductor circuits may also be formed on both sides of the semiconductor device.

Since in the image pickup apparatus 1E, an image pickup signal outputted from the image pickup device 20 is subjected to primary processing before being transmitted, the signal is less likely to be degraded.

Note that image pickup sections of the image pickup apparatuses 1, 1A to 1D may be stacked semiconductors in which a plurality of semiconductor devices including image pickup devices are stacked. For example, optical sections of the image pickup apparatuses 1, 1A to 1C and 1E may be lens units including a plurality of lenses.

It goes without saying that endoscopes 9A to 9E including the image pickup apparatuses 1A to 1E include effects of the endoscope 9 and effects of the image pickup apparatuses 1A to 1E.

The present invention is not limited to the aforementioned embodiments or the like, and various changes and modifications can be made without departing from the gist of the present invention.

What is claimed is:

1. An image pickup apparatus comprising:
a wiring board including a mounting surface;
an image pickup component mounted on the mounting surface, a light-receiving section being disposed on a light-receiving surface of the image pickup component;
an optical component including a first main surface and a second main surface on an opposite side of the first main surface, the second main surface being bonded to the light-receiving surface;
an electronic component mounted on the mounting surface, the electronic component being a semiconductor device, a passive device, or an illumination component; and
sealing resin disposed on the mounting surface, covering the image pickup component, located on a same surface as the first main surface and including a resin upper surface parallel to the mounting surface.

2. The image pickup apparatus according to claim 1, wherein the electronic component is a chip capacitor, a chip inductor, a chip resistor, or an integrated circuit.

3. The image pickup apparatus according to claim 1, wherein
the illumination component comprises a light-emitting element and a light guide component,
the light-emitting element includes a light-emitting surface and a rear surface on an opposite side of the light-emitting surface, the light guide component includes a third main surface and a fourth main surface on an opposite side of the third main surface, and the fourth main surface is bonded to the light-emitting surface,
the light-emitting surface and the light-receiving surface are on different surfaces, and
the third main surface is on a same surface as the first main surface and the resin upper surface.

4. The image pickup apparatus according to claim 1, wherein the optical component is a cover glass.

5. The image pickup apparatus according to claim 1, wherein the optical component is a lens unit in which a plurality of optical elements including a cover glass are stacked.

6. The image pickup apparatus according to claim 1, wherein the image pickup component is an image pickup device or a stacked semiconductor in which a plurality of semiconductor devices including the image pickup device are stacked.

7. The image pickup apparatus according to claim 1, wherein the first main surface and the resin upper surface are polished surfaces.

8. An endoscope comprising an image pickup apparatus, the image pickup apparatus comprising:
a wiring board including a mounting surface;
an image pickup component mounted on the mounting surface, a light-receiving section being disposed on a light-receiving surface of the image pickup component;
an optical component including a first main surface and a second main surface on an opposite side of the first main surface, the second main surface being bonded to the light-receiving surface;
an electronic component mounted on the mounting surface, the electronic component being a semiconductor device, a passive device, or an illumination component; and
sealing resin disposed on the mounting surface, covering the image pickup component, located on a same surface as the first main surface and including a resin upper surface parallel to the mounting surface.

9. A method for manufacturing an image pickup apparatus, comprising:
mounting an image pickup component with an optical component bonded to a light-receiving surface, and an electronic component, which is a semiconductor device, a passive device, or an illumination component, on a mounting surface of a wiring board;
disposing sealing resin on the mounting surface, the sealing resin covering the image pickup component and the optical component; and
polishing the sealing resin to thereby expose the optical component to a polished surface and polishing the optical component exposed to the polished surface and the sealing resin simultaneously.

10. The method for manufacturing an image pickup apparatus according to claim 9, wherein polishing ends when a component upper surface of the electronic component is exposed to the polished surface.

11. The method for manufacturing an image pickup apparatus according to claim 9, wherein
the illumination component comprises a light-emitting element and a light guide component,
the light-emitting element includes a light-emitting surface and a rear surface on an opposite side of the light-emitting surface, the illumination component is bonded to the light-emitting surface, a height from the mounting surface of the light-emitting surface is different from a height of the light-receiving surface,
the illumination component is covered with the sealing resin, and
the light guide component exposed to the polished surface is polished.

12. The method for manufacturing an image pickup apparatus according to claim 9, wherein
a wafer process is performed up to polishing, and
a wafer comprising a plurality of image pickup components and a plurality of optical components is divided into individual pieces.
